(19) United States
(12) Patent Application Publication
(10) Pub. No.: US 2007/0276027 A1
(43) Pub. Date: Nov. 29, 2007

(54) Crystalline Forms of [R-(R* ,R*)]-2-(4-Fluorophenyl)-Beta, -Dihydroxy-5-(1-Methylethyl)-3-Phenyl-4-[(Phenylamino)Carbonyl]-1H-Pyrrole-1-Heptanoic Acid

(75) Inventors: Anthony Michael Campeta, Ledyard, CT (US); Joseph Francis Krzyzaniak, Pawcatuck, CT (US); Jason Albert Leonard, Concord, NH (US)

Correspondence Address: WARNER-LAMBERT COMPANY, 2800 PLYMOUTH RD, ANN ARBOR, MI, 48105, US

(73) Assignee: Warner-Lambert Company LLC

(21) Appl. No.: 10/572,324

(22) PCT Filed: Sep. 6, 2004
(86) PCT No.: PCT/IB04/02919

§ 371 Date: Apr. 18, 2007

Related U.S. Application Data
(60) Provisional application No. 60/503,592, filed on Sep. 17, 2003.

(51) Int. Cl. C07D 207/34; A61P 3/06

(52) U.S. Cl. 514/423; 548/537

(57) ABSTRACT

Novel crystalline forms of [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid (atorvastatin free acid) designated Form A and Form B, characterized by their X-ray powder diffraction pattern and solid-state NMR spectra are described, as well as methods for the preparation and pharmaceutical composition of the same, which are useful as agents for treating hyperlipidemia, hypercholesterolemia, osteoporosis, benign prostatic hyperplasia, and Alzheimer's Disease, and for preparing salts of atorvastatin.

### FIELD OF THE INVENTION

The present invention relates to novel crystalline forms of atorvastatin free acid which is known by the chemical name [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, useful as intermediates to prepare pharmaceutically acceptable salts of atorvastatin, including atorvastatin calcium, and useful as pharmaceutical agents, to methods for their production and isolation to pharmaceutical compositions which include these compounds and a pharmaceutically acceptable carrier, as well as methods of using such compositions to treat subjects, including human subjects, suffering from hyperlipidemia, hypercholesterolemia, benign prostatic hyperplasia, osteoporosis, and Alzheimer's Disease.

### BACKGROUND OF THE INVENTION

The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. As such, statins are collectively potent lipid lowering agents.

Atorvastatin calcium is currently sold as Lipitor® having the chemical name [R—(R*,R*)]-2-(4-fluorophenyl)-β,δ-hydroxy-5-(1-methylethyl)-3-phenyl-4-[(phenylamino)carbonyl]-1H-pyrrole-1-heptanoic acid calcium salt (2:1) trihydrate and the formula:

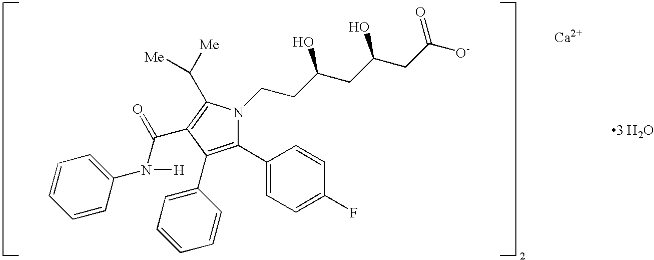

The nonproprietary name designated by USAN (United States Adopted Names) is atorvastatin calcium and by INN (International Nonproprietary Name) is atorvastatin. Under the established guiding principles of USAN, the salt is included in the name whereas under INN guidelines, a salt description is not included in the name.

Atovastatin and pharmaceutically acceptable salts thereof are selective, competitive inhibitors of HMG-CoA reductase. As such, atorvastatin calcium is a potent lipid lowering compound and is thus useful as a hypolipidemic and/or hypocholesterolemic agent, as well as in the treatment of osteoporosis, benign prostatic hyperplasia, and Alzheimer's disease.

A number of patents have issued disclosing atorvastatin calcium, formulations of atorvastatin calcium, as well as processes and key intermediates for preparing atorvastatin calcium. These include: U.S. Pat. Nos. 4,681,893; 5,273,995; 5,003,080; 5,097,045; 5,103,024; 5,124,482; 5,149,837; 5,155,251; 5,216,174; 5,245,047; 5,248,793; 5,280,126; 5,397,792; 5,342,952; 5,298,627; 5,446,054; 5,470,981; 5,489,690; 5,489,691; 5,510,488; 5,686,104; 5,998,633; 6,087,511; 6,126,971; 6,433,213; and 6,476,235, which are herein incorporated by reference.

Atorvastatin calcium can exist in crystalline, liquid-crystalline, non-crystalline and amorphous forms.

Crystalline forms of atorvastatin calcium are disclosed in U.S. Pat. Nos. 5,969,156 and 6,121,461 which are herein incorporated by reference. Further crystalline, liquid crystalline, plastic crystalline, disordered forms and non-crystalline forms, as well as mesophases are disclosed in copending applications: Published International Patent Application WO 03/004470 and U.S. Patent Application Ser. No. 60/414,734, which are herein incorporated by reference.

Additionally, a number of published International Patent Applications have disclosed crystalline forms of atorvastatin calcium, as well as processes for preparing amorphous atorvastatin calcium. These include: WO 00/71116; WO 01/28999; WO 01/36384; WO 01/42209; WO 02/41834; WO 02/43667; WO 02/43732; WO 02/051804; WO 02/057228; WO 02/057229; WO 02/057274; WO 02/059687; WO 02/072073; WO 02/083637; WO 02/083638; and WO 02/089788.

Atorvastatin is prepared as its calcium salt, i.e., [R—(R*,R*)]-2-(4 fluorophenyl)-β,δ-dihydroxy-5-(1-methylethyl)-3-phenyl-4-[(phenylamino)carbonyl]-1H-pyrrole-1-1-heptanoic acid calcium salt (2:1). The calcium salt is desirable since it enables atorvastatin to be conveniently formulated in, for example, tablets, capsules, lozenges, powders, and the like for oral administration.

Atorvastatin free acid, disclosed in U.S. Pat. No. 5,213,995, can be used to prepare the calcium salt of atorvastatin, as well as other pharmaceutically acceptable basic addition salts of atorvastatin. Additionally, atorvastatin free acid can be used as a pharmaceutical agent. However, prior to the present invention, atorvastatin free acid could only be isolated as an oil. Therefore, there was a need to prepare atorvastatin free acid in solid, preferably crystalline, form to facilitate the preparation of salts of atorvastatin, as well as pharmaceutical compositions containing the free acid of atorvastatin.

We have now surprisingly and unexpectedly found novel crystalline forms of atorvastatin free acid. Thus, the present invention provides atorvastatin free acid in new crystalline forms designated Forms A and B. The new crystalline forms of atorvastatin free acid are purer, more stable, and have advantageous properties compared to the prior noncrystalline form.

### SUMMARY OF THE INVENTION

Accordingly, a first aspect of the present invention is directed to crystalline forms of atorvastatin free acid and hydrates thereof.

In a second aspect, the invention is directed to crystalline Form A atorvastatin free acid and hydrates thereof characterized by the following x-ray powder diffraction pattern expressed in terms of the 2θ, d-spacings, and relative intensities with a relative intensity of >20% measured on a Bruker D5000 diffractometer with CuKα radiation:

| | | Relative* Intensity |
|---|---|---|
| Degree 2θ | d (Å) | (>20%) |
| 4.7 | 18.7 | 49.5 |
| 6.0 | 14.6 | 25.9 |
| 8.9 | 9.9 | 46.0 |
| 9.1 | 9.8 | 63.0 |
| 9.4 | 9.4 | 100.0 |
| 13.2 | 6.7 | 20.5 |
| 14.1 | 6.3 | 29.5 |
| 17.8 | 5.0 | 55.8 |
| 18.1 | 4.9 | 98.1 |
| 18.9 | 4.7 | 63.8 |
| 19.9 | 4.5 | 23.9 |
| 20.2 | 4.4 | 29.3 |
| 20.6 | 4.3 | 32.4 |
| 21.8 | 4.1 | 50.1 |
| 22.1 | 4.0 | 57.5 |
| 22.5 | 4.0 | 28.4 |
| 23.7 | 3.8 | 57.1 |
| 25.9 | 3.4 | 21.0 |
| 26.7 | 3.3 | 20.0 |

*The relative intensities may change depending on the crystal size and morphology.

Further, in a third aspect, the present invention is directed to crystalline Form A atorvastatin free acid and hydrates thereof characterized by the following solid-state ¹³C nuclear magnetic resonance (SSNMR) spectrum wherein chemical shift is expressed in parts per million (ppm):

| Assignment | Carbon chemical shift (ppm)* |
|---|---|
| C39 | 180.6 |
| C39 | 174.3 |
| C 8 | 167.1 |
| C 8 | 166.3 |
| C27 | 163.6 |
| C27 | 161.5 |
| Following group of resonances include: | |
| C1, 2, 3, 4, 6, 7, 9, 10, 12, 13, 17, 18, | 141.8 |
| 20, 21, 24, 25, 28, 29, 33, 34, 36 | 140.7 |
| | 137.9 |
| | 135.2 |
| | 134.1 |
| | 132.9 |
| | 130.0 |
| | 128.8 (shoulder) |
| | 128.0 |
| | 125.4 |
| | 123.3 |
| | 121.6 |
| | 119.3 |
| | 118.4 |
| | 116.4 |
| | 115.1 |
| | 113.7 |
| | 112.3 |
| Following group of resonances include: | |
| C26, 35 | 71.3 |
| | 70.0 |
| | 69.1 |
| | 68.6 |
| | 65.3 |
| Following group of resonances include: | |
| C11, 19, 30, 37 | 43.5 |
| | 42.9 |
| | 41.8 |
| | 40.6 |
| | 40.0 |
| | 38.9 |
| | 37.1 |
| Following group of resonances include: | |
| C14, 22, 23 | 26.8 |
| | 26.2 |
| | 25.5 |
| | 25.0 |
| | 21.2 |
| | 20.5 |
| | 18.8 |
| | 18.1 |

Peak at 8.4 ppm is a spinning side band
*Values in ppm with respect to tetramethylsilane (TMS) at 0 ppm; referenced using an external sample of adamantane, setting is upfield resonance to 29.5 ppm.

Additionally, in a fourth aspect, the present invention is directed to crystalline Form A atorvastatin free acid and hydrates thereof characterized by the following solid-state ¹⁹F nuclear magnetic resonance spectrum wherein chemical shift is expressed in parts per million:

| Assignment | Flourine chemical shift (ppm)* |
|---|---|
| F | −105.6 |
| | −110.6 |
| | −112.6 |
| | −114.1 |

*Values in ppm with respect to CCl₃F at 0 ppm; referenced by setting ¹⁹F signal of trifluoroacetic acid (TFA) —H₂O (1:1) to −76.54 ppm.

In a fifth aspect, the present invention is directed to crystalline Form B atorvastatin free acid and hydrates thereof characterized by the following x-ray powder diffraction pattern expressed in terms of the 2θ, d-spacings, and relative intensities with a relative intensity of >20% measured on a Bruker D5000 diffractometer with CuKα radiation:

| | | Relative* Intensity |
|---|---|---|
| Degree 2θ | d (Å) | (>20%) |
| 4.6 | 19.0 | 48.3 |
| 5.9 | 15.0 | 32.4 |
| 8.6 | 10.2 | 46.6 |
| 9.3 | 9.5 | 100.0 |
| 13.3 | 6.6 | 33.7 |
| 14.1 | 6.3 | 33.4 |
| 17.4 | 5.1 | 46.7 |
| 17.7 | 5.0 | 43.1 |
| 18.0 | 4.9 | 77.0 |
| 18.8 | 4.7 | 66.4 |
| 19.3 | 4.6 | 21.5 |
| 19.8 | 4.5 | 23.5 |
| 20.2 | 4.4 | 21.5 |
| 21.1 | 4.2 | 36.7 |
| 21.5 | 4.1 | 38.3 |
| 21.9 | 4.1 | 31.6 |
| 23.6 | 3.8 | 44.8 |

*The relative intensities may change depending on the crystal size and morphology.

As inhibitors of HMG-CoA reductase, the novel crystalline forms of atorvastatin free acid are useful as hypolipidemic and hypocholesterolemic agents, as well as agents in the treatment of osteoporosis, benign prostatic hyperplasia, and Alzheimer's Disease.

A still further embodiment of the present invention is a pharmaceutical composition for administering an effective amount of crystalline Form A or Form B atorvastatin free acid in unit dosage form in the treatment methods mentioned above. Finally, the present invention is directed to methods for production of Forms A and B atorvastatin free acid.

### BRIEF DESCRIPTION OF THE DRAWINGS

The invention is further described by the following nonlimiting examples which refer to the accompanying FIGS. 1 to 4, short particulars of which are given below.

FIG. 1

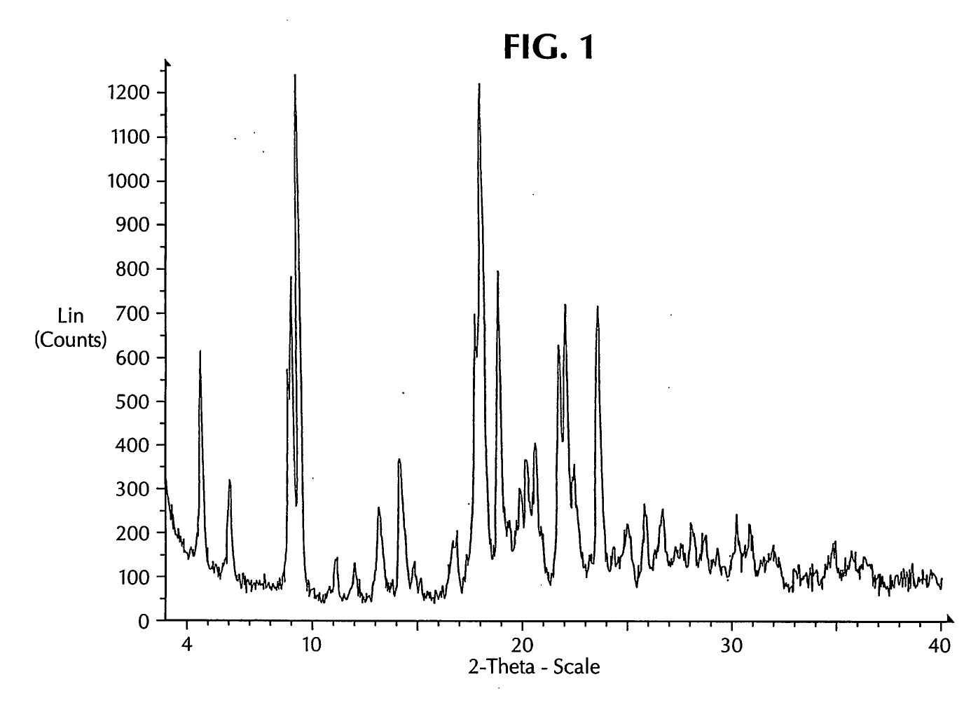

Diffractogram of Form A atorvastatin free acid carried out on a Bruker D5000 diffractometer.

FIG. 2

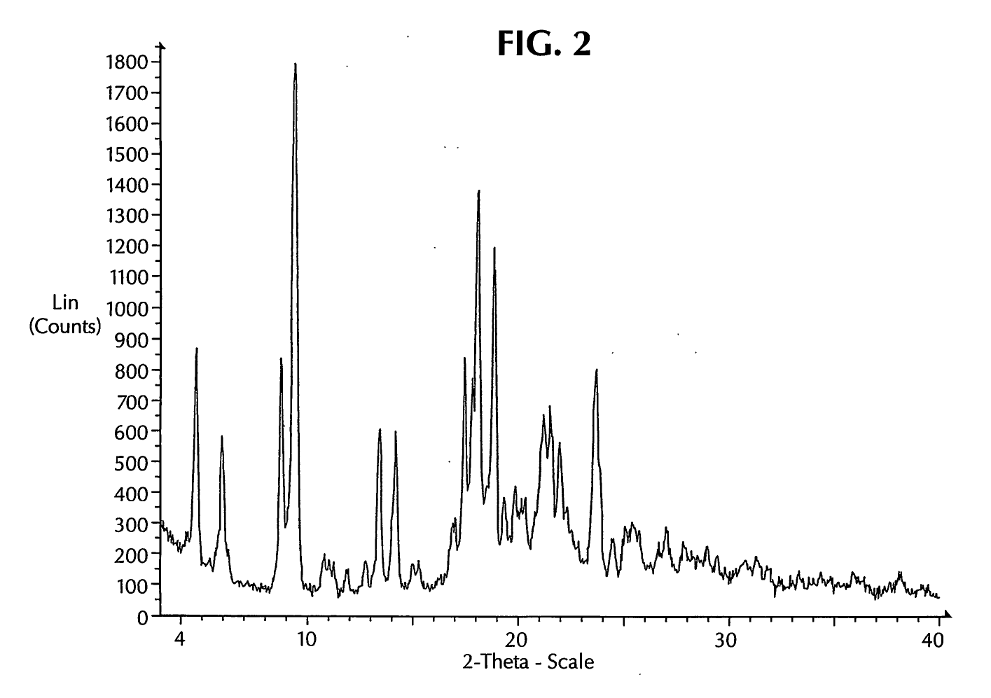

Diffractogram of Form B atorvastatin free acid carried out on a Bruker D5000 diffractometer.

FIG. 3

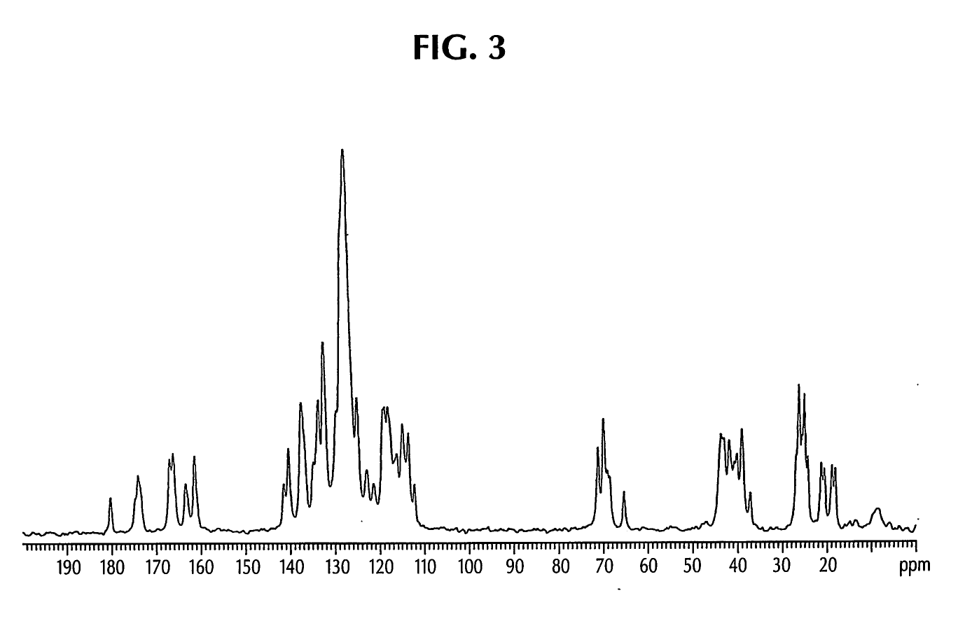

Solid-state ¹³C nuclear magnetic resonance spectrum of Form A atorvastatin free acid.

FIG. 4

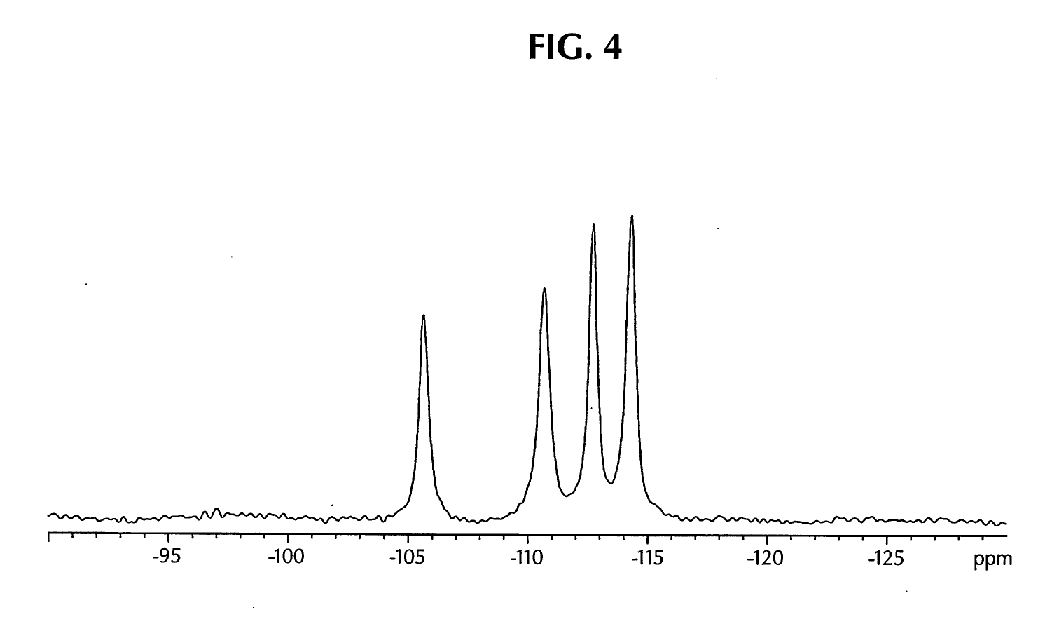

Solid-state ¹⁹F nuclear magnetic resonance spectrum of Form A atorvastatin free acid.

### DETAILED DESCRIPTION OF THE INVENTION

The term “crystalline” as used herein refers to a solid formed by a repeating three-dimensional pattern of atoms, ions or molecules and having fixed distances between the constituent parts and furthermore, can be identified by one skilled in the art using methods, such as, for example x-ray diffraction, solid-state NMR, Raman spectroscopy, Infrared spectroscopy and the like. Examples of crystalline solids disclosed in the present application include crystalline Form A and Form B atorvastatin free acid. Crystalline Form A and Form B atorvastatin free acid may be characterized by their x-ray powder diffraction patterns and/or by their solid state nuclear magnetic resonance spectra.

### Powder X-Ray Diffraction

Forms A and B atorvastatin free acid were characterized by their powder x-ray diffraction patterns. Thus, the x-ray diffraction patterns of Forms A and B were carried out on a Bruker D5000 diffractometer using copper radiation (wavelength 1:1.54056. The tube voltage and amperage were set to 40 kV and 50 mA, respectively. The divergence and scattering slits were set at 1 mm, and the receiving slit was set at 0.6 mm. Diffracted radiation was detected by a Kevex PSI detector. A theta-two theta continuous scan at 2.4°/min (1 sec/0.04° step) from 3.0 to 40° 2θ was used. An alumina standard was analyzed to check the instrument alignment. Data were collected and analyzed using Bruker axis software Version 7.0. Samples were prepared by placing them in a quartz holder. It should be noted that Bruker Instruments purchased Siemans; thus, Bruker D5000 instrument is essentially the same as a Siemans D5000.

Table 1 lists the 20 and relative intensities of all lines that have a relative intensity of >20% in the sample for crystalline Forms A and B atorvastatin free acid:

TABLE 1
INTENSITIES AND PEAK LOCATIONS OF DIFFRACTION LINES
FOR ATORVASTATIN FREE ACID, FORMS A AND B

| | |
|---|---|
| FORM A | FORM B |

| Degree | Relative Intensity | Degree | Relative Intensity |
|---|---|---|---|
| 2θ | (>20%) | 2θ | (>20%) |
| 4.7 | 49.5 | 4.6 | 48.3 |
| 6.0 | 25.9 | 5.9 | 32.4 |
| 8.9 | 46.0 | 8.6 | 46.6 |
| 9.1 | 63.0 | 9.3 | 100.0 |
| 9.4 | 100.0 | 13.3 | 33.7 |
| 13.2 | 20.5 | 14.1 | 33.4 |
| 14.1 | 29.5 | 17.4 | 46.7 |
| 17.8 | 55.8 | 17.7 | 43.1 |
| 18.1 | 98.1 | 18.0 | 77.0 |
| 18.9 | 63.8 | 18.8 | 66.4 |
| 19.9 | 23.9 | 19.3 | 21.5 |
| 20.2 | 29.3 | 19.8 | 23.5 |
| 20.6 | 32.4 | 20.2 | 21.5 |
| 21.8 | 50.1 | 21.1 | 36.7 |
| 22.1 | 57.5 | 21.5 | 38.3 |
| 22.5 | 28.4 | 21.9 | 31.6 |
| 23.7 | 57.1 | 23.6 | 44.8 |
| 25.9 | 21.0 | | |
| 26.7 | 20.0 | | |

Because only two crystalline forms of atorvastatin free acid are known, each form can be identified and distinguished from the other crystalline form by either a single x-ray powder diffraction line, a combination of lines or a pattern that is different from the x-ray powder diffraction of the other form.

For example, Table 2 lists single unique 2θ peaks for Forms A and B atorvastatin free acid, i.e., a set of x-ray diffraction lines that are unique to each form.

TABLE 2
ATORVASTATIN FREE ACID, FORMS A AND B UNIQUE
PEAKS AND COMBINATIONS OF 2θ PEAKS

| FORM A | FORM B |
|---|---|
| Degree 2θ | Degree 2θ |
| 8.9 | 8.6 |
| 20.6 | 17.4 |
| 22.5 | 21.1 |
| 25.9 | 21.5 |

### Solid State Nuclear Magnetic Resonance

Form A atorvastatin free acid may also be characterized by its solid-state nuclear magnetic resonance spectra. Thus, the solid-state nuclear magnetic resonance spectra of Form A was carried out on a Bruker-Biospin Avance DSX 500 MHz NMR spectrometer.

¹⁹F SSNMR

Approximately 15 mg of sample were tightly packed into a 2.5 mm ZrO spinner for each sample analyzed. One-dimensional ¹⁹F spectra were collected at 295 K and ambient pressure on a Bruker-Biospin 2.5 mm BL cross-polarization magnetic angle spinning (CPMAS) probe positioned into a wide-bore Bruker-Biospin Avance DSX 500 MHz NMR spectrometer. The samples were positioned at the magic angle and spun at 35.0 kHz, corresponding to the maximum specified spinning speed for the 2.5 mm spinners. The fast spinning speed minimized the intensities of the spinning side bands and provided almost complete decoupling of ¹⁹F signals from protons. The number of scans were individually adjusted for each sample to obtain adequate single/noise (S/N). Typically, 150 scans were acquired. Prior to ¹⁹F acquisition, ¹⁹F relaxation times were measured by an inversion recovery technique. The recycle delay for each sample was then adjusted to five times the longest ¹⁹F relaxation time in the sample, which ensured acquisition of quantitative spectra. A background due to probe ringing was subtracted in each alternate scan after presaturating the ¹⁹F signal. The spectra were referenced using an external sample of trifluoroacetic acid (diluted to 50% V/V by H₂O), setting its resonance to −76.54 ppm.

¹³C SSNMR

Approximately 80 mg of sample were tightly packed into a 4 mm ZrO spinner for each sample analyzed. One-dimensional ¹³C spectra were collected at ambient pressure using ¹H-¹³C CPMAS at 295 K on a Bruker 4 mm BL CPMAS probe positioned into a wide-bore Bruker-Biospin Avance DSX 500 MHZ NMR spectrometer. The samples were spun at 15.0 kHz corresponding to the maximum specified spinning speed for the 4 mm spinners. The fast spinning speed minimized the intensities of the spinning side bands. To optimize the signal sensitivity, the cross-polarization contact time was adjusted to 1.5 ms, and the proton decoupling power was set to 100 kHz. The number of scans were individually adjusted for each sample to obtain adequate S/N. Typically, 1900 scans were acquired with a recycle delay of 5 seconds. The spectra were referenced using an external sample of adamantane, setting its upfield resonance at 29.5 ppm.

Atorvastatin free acid crystalline Forms A and B of the present invention may exist in anhydrous forms as well as hydrated and solvated forms. In general, the hydrated forms are equivalent to unhydrated forms and are intended to be encompassed within the scope of the present invention. Crystalline Form A preferably occurs as a hydrate. Preferably, Form A contains 0.6 mol of water.

Atorvastatin free acid crystalline Forms A and B of the present invention, regardless of the extent of hydration and/or solvation having equivalent x-ray powder diffractograms, or SSNMR, are within the scope of the present invention.

The new crystalline forms of atorvastatin free acid described herein have advantageous properties. For example, Forms A and B have good chemical stability. Also, the solubility of Forms A and B in solvents including water and phosphate buffered saline solution are comparable to Form I atorvastatin calcium (disclosed in U.S. Pat. No. 5,969,156).

The present invention provides a process for the preparation of crystalline Forms A and B atorvastatin free acid which comprises crystallizing atorvastatin free acid from a solution in solvents under conditions which yield crystalline Forms A and B atorvastatin free acid.

The precise conditions under which crystalline Forms A and B atorvastatin free acid are formed may be empirically determined, and it is only possible to give a number of methods which have been found to be suitable in practice.

For example, Form A can be prepared by slurrying atorvastatin calcium in water with a solvent such as, for example, acetonitrile and the like. The mixture is filtered and the filtrate is acidified with an acid such as, for example, an inorganic acid such as hydrochloric acid and the like, followed by removal of the solvent. The solid is washed with water and dried to afford Form A. Preferably, crystalline Form I atorvastatin calcium is slurried in a mixture of about 80 parts of water and 20 parts of acetonitrile, the mixture is filtered, the filtrate is acidified with 1N HCl, the solvent removed, the resulting solid washed with water and dried at about room temperature for about 24 hours to afford Form A.

Alternatively, Form A may be prepared by solvent extraction. For example, atorvastatin calcium is slurried in water until wet, followed by the addition of a solvent such as, for example, methyl tertiary butyl ether (MTBE), ethyl acetate and the like. The suspension is acidified with an acid as disclosed above, stirred until a clear two phase mixture results, the organic phase is separated, the solvent removed, and the resulting solid is dissolved in a solvent such as water and acetonitrile to afford Form A. Seeds of Form A can be added after the solid is dissolved in water-acetonitrile to accelerate the formation of Form A. Preferably, crystalline Form I atorvastatin calcium is slurried in a mixture of water and MTBE, the suspension is acidified with 1N HCl, the two phases are separated, the MTBE is removed, the resulting solid is dissolved in water-acetonitrile, seeds of Form A are added and Form A is isolated by filtration.

Form B is prepared by heating Form A at about 45° C. under vacuum for about one day. Preferably, Form A is heated in a oven at about 45° C. under vacuum for about one day. Alternatively, Form A is exposed to low relative humidity for about 72 days to afford Form B. Preferably, Form A is stored in a low relative humidity chamber prepared using phosphorous pentoxide for about 72 days to afford Form B.

The compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds of the present invention can be administered by inhalation, for example, intranasally. Additionally, the compounds of the present invention can be administered transdermally.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from two or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, retention enemas, and emulsions, for example water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.5 mg to 100 mg, preferably 2.5 mg to 80 mg according to the particular application and the potency of the active component. The composition can, if desired, also contain other compatible therapeutic agents.

In therapeutic use as hypolipidemic and/or hypocholesterolemic agents and agents to treat osteoporosis, benign prostatic hyperplasia, and Alzheimer's disease, crystalline Forms A and B atorvastatin free acid utilized in the pharmaceutical method of this invention are administered at the initial dosage of about 2.5 mg to about 80 mg daily. A daily dose range of about 2.5 mg to about 20 mg is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstance is reached. For convenience, the total daily dosage may be divided and administered in portions during the day if desired.

The following nonlimiting examples illustrate the inventors' preferred methods for preparing the compounds of the invention.

### EXAMPLE 1

### [R—(R*,R*)]-2-(4-Fluorophenyl)-β,δ-dihydroxy-5-(1-methylethyl)-3-phenyl-4-[(phenylamino)carbonyl]-1H-pyrrole-1-heptanoic acid

Form A Atorvastatin Free Acid

Method A

In a 600 mL beaker, a slurry was prepared by charging 100 mL of acetonitrile (ACN) and 400 mL deionized water (20:80 ACN:water) to 0.5 grams of crystalline Form I atorvastatin calcium (U.S. Pat. No. 5,969,156). The slurry was stirred at ambient conditions for 15 minutes. All undissolved material was removed by vacuum filtration using a 0.45 μm nylon-66 membrane filter. The pH of the filtrate was determined to be 6.5-7.0, which was then adjusted to pH 2.35 with 1N HCl. A cloudy precipitate formed and determined by PLM to be fine droplets of oil. Solvent was evaporated by passing nitrogen over the headspace of the solution with stirring until a heavy white precipitate formed (˜15 minutes). The slurry was vacuum filtered through a 0.45 μm nylon-66 membrane filter. The solids were then washed with 100 mL of deionized water and air dried at ambient conditions for 24 hours to afford 0.3 grams of crystalline Form A atorvastatin free acid.

Method B

Crystalline Form I atorvastatin calcium (U.S. Pat. No. 5,969,156) (10 grams) was placed in an Erlenmeyer flask (4 L). Water (1 L) was added to the flask along with a magnetic stir bar. The contents were stirred until all of the solids were wet. With stirring, MTBE (methyl tert-butyl ether-1 L) was added to the reaction mixture to form a white suspension. Hydrochloric Acid (20 mL-1 N solution) was then added to the suspension with stirring. The contents were stirred until a clear mixture (2 phases) was present (ca. 5 min). The mixture was then transferred into a separatory funnel (4 L). The contents were mixed well, and the layers separated. The water layer (lower phase) was placed back into the separatory funnel and additional MTBE (1 L) was added. The contents were mixed well, and the layers were separated. The water layer was discarded, and the MTBE layer was combined with the MTBE layer from the first extraction. The combined MTBE layers were placed back into the funnel and water was added (0.5 L). The contents were mixed well, and the layers were separated. The water layer was discarded, and the MTBE layer was placed into a round-bottomed flask (3 L). The MTBE was then removed via rotary evaporation producing a thin film or amorphous solid. The film/solid was dissolved with acetonitrile (0.2 L) to form a solution. Water (0.8 L) was added to the solution with stirring using a magnetic stir bar. A white suspension was formed that appeared as oil droplets by PLM (polarized-light microscopy). Seed crystals of Form A atorvastatin free acid were added. The contents were then rapidly stirred under a nitrogen bleed for approximately one hour. The solids were isolated by vacuum filtration using a Büchner funnel fitted with a paper filter (#2). The solids were rinsed using water (0.5 L), and placed in a crystallizing dish. The dish was placed in an oven at 25° C. maintaining nitrogen until dry (ca. 1 day). This procedure afforded crystalline Form A atorvastatin free acid in a yield of approximately 92%.

Form B Atorvastatin Free Acid

Method A

Crystalline Form A atrovastatin free acid (Example 1) was stored in a vacuum oven at 45° C. (nitrogen purge, house vacuum) for about one day to afford crystalline Form B atorvastatin free acid.

Method B

Crystalline Form A atorvastatin free acid (Example 1) was stored in a low relative humidity chamber (prepared using phosphorous pentoxide) for about 72 days to afford crystalline Form B atorvastatin free acid.

1. A crystalline atrovastatin free acid.

2. A crystalline Form A atorvastatin free acid or a hydrate thereof having a X-ray powder diffraction pattern containing the following 2θ values measured using CuK_{α} radiation: 8.9, 20.6, 22.5, or 25.9.

3. A crystalline Form A atorvastatin free acid or a hydrate thereof having a X-ray powder diffraction pattern containing the following 2θ values measured using CuK_{α} radiation: 4.7, 6.0, 8.9, 9.1, 9.4, 13.2, 14.1, 17.8, 18.1, 18.9, 19.9, 20.2, 20.6, 21.8, 22.1, 22.5, 23.7, 25.9, and 26.7.

4. A crystalline Form A atorvastatin free acid having a X-ray powder diffraction pattern containing the following 2θ values measured using CuK_{α} radiation: 8.9, 20.6, 22.5, or 25.9.

5. A crystalline Form A atorvastatin free acid having a X-ray powder diffraction pattern containing the following 2θ values measured using CuK_{α} radiation: 4.7, 6.0, 8.9, 9.1, 9.4, 13.2, 14.1, 17.8, 18.1, 18.9, 19.9, 20.2, 20.6, 21.8, 22.1, 22.5, 23.7, 25.9, and 26.7.

6. A crystalline Form A atorvastatin free acid or a hydrate thereof characterized by solid-state ¹³C nuclear magnetic resonance having the following chemical shifts expressed in parts per million: 18.1, 18.8, 20.5, and 21.2.

7. A crystalline Form A atorvastatin free acid or a hydrate thereof characterized by solid-state ¹³C nuclear magnetic resonance having the following chemical shifts expressed in parts per million: 161.5, 163.6, 166.3, 167.1, 174.3, and 180.6.

8. A crystalline Form A atorvastatin free acid or a hydrate thereof characterized by solid-state ¹³C nuclear magnetic resonance having the following chemical shifts expressed in parts per million: 18.1, 18.8, 20.5, 21.2, 25.0, 25.5, 26.2, 26.8, 37.1, 38.9, 40.0, 40.6, 41.8, 42.9, 43.5, 65.3, 68.6, 69.1, 70.0, 71.3, 112.3, 113.7, 115.1, 116.4, 118.4, 119.3, 121.6, 123.3, 125.4, 128.0, 128.8 (shoulder), 130.0, 132.9, 134.1, 135.2, 137.9, 140.7, 141.8, 161.5, 163.6, 166.3, 167.1, 174.3, and 180.6.

9. A crystalline Form A atorvastatin free acid or a hydrate thereof characterized by solid-state ¹⁹F nuclear magnetic resonance having the following chemical shifts expressed in parts per million: −114.1, −112.6, −110.6, and −105.6.

10. A crystalline Form A atorvastatin free acid characterized by solid-state ¹⁹F nuclear magnetic resonance having the following chemical shifts expressed in parts per million: −114.1, −12.6, −10.6, Of and −105.6.

11. A crystalline Form B atorvastatin free acid or a hydrate thereof having a X-ray powder diffraction pattern containing the following 2θ values measured using CuK_{α} radiation: 8.6, 17.4, 21.1, and 21.5.

12. A crystalline Form B atorvastatin free acid or a hydrate thereof having a X-ray powder diffraction pattern containing the following 2θ values measured using CuK_{α} radiation: 4.6, 5.9, 8.6, 9.3, 13.3, 14.1, 17.4, 17.7, 18.0, 18.8, 19.3, 19.8, 20.2, 21.1, 21.5, 21.9, and 23.6.

13. A crystalline Form B atorvastatin free acid having a X-ray powder diffraction pattern containing the following 2θ values measured using CuK_{α} radiation: 4.6, 5.9, 8.6, 9.3, 13.3, 14.1, 17.4, 17.7, 18.0, 18.8, 19.3, 19.8, 20.2, 21.1, 21.5, 21.9, and 23.6.

14. A pharmaceutical composition comprising crystalline atrovastatin free acid in admixture with at least one pharmaceutically acceptable excipient, diluent, or carrier.

15. A method of treating hyperlipidemia, hypercholesterolemia, osteoporosis, benign prostatic hyperplasia, and Alzheimer's Disease comprising administering to a host suffering therefrom a therapeutically effective amount of a compound according to claim 1 in unit dosage form.

16. A crystalline atorvastatin free acid hydrate.
